(19) United States
(12) Patent Application Publication
(10) Pub. No.: US 2016/0038446 A1
(43) Pub. Date: Feb. 11, 2016

(54) METHOD FOR IMPEDING SKIN INFLAMMATORY RESPONSE TO AN ENERGY INJURY

(71) Applicant: James Beckman, Springdale, AR (US)

(72) Inventor: James Beckman, Springdale, AR (US)

(21) Appl. No.: 14/454,331

(22) Filed: Aug. 7, 2014

(51) Int. Cl. A61K 31/19; A61K 45/06; A61K 9/00

(52) U.S. Cl. CPC A61K 31/19; A61K 9/0014; A61K 45/06

(57) ABSTRACT

A method for treating skin that is subject to an energy injury including the application of concentrated l-lactic acid to the injury area during the inflammatory response of the skin. Reapplication is shown to further reduce the inflammatory symptoms and improve healing time. The concentration of approximately ten percent by weight with around 2.34 pH is proven to be ideal to impede the skin's inflammatory response for sunburn and elevated temperature thermal burns and prevent associated cellular damage compared to untreated sunburns.

### CROSS-REFERENCE TO RELATED APPLICATIONS

This application claims priority to and is a continuation-in-part of U.S. Provisional Application Ser. No. 61/863,634 filed on Aug. 8, 2013 by Beckman entitled Fruit Acid Sunburn Treatment.

### STATEMENT REGARDING FEDERALLY SPONSORED RESEARCH OR DEVELOPMENT

Not Applicable.

### REFERENCE TO A MICROFICHE APPENDIX

Not Applicable.

### RESERVATION OF RIGHTS

A portion of the disclosure of this patent document contains material which is subject to intellectual property rights such as but not limited to copyright, trademark, and/or trade dress protection. The owner has no objection to the facsimile reproduction by anyone of the patent document or the patent disclosure as it appears in the Patent and Trademark Office patent files or records but otherwise reserves all rights whatsoever.

### BACKGROUND OF THE INVENTION

1. Field of the Invention

The present invention relates to improvements in medical treatment for injuries. More particularly, the invention relates to improvements particularly suited for treating skin injuries such as sunburn that have an inflammatory response.

2. Description of the Known Art

a. Skin Injury Response

As will be appreciated by those skilled in the art, skin injuries are known in various forms. When an injury of any type of occurs, the human body automatically responds by initiating a chain reaction of events or a series of reactions that is called the inflammatory process. These reactions occur both within the injured cells and in locations outside the cells and involve cascades of cellular responses.

All injuries to the human body create or result in a wound. This is easily understandable to non-medical persons in reference to cuts into the skin (including surgical operations), penetrating injuries with foreign objects, crush injuries, broken bones, etc. and other forms of trauma from mechanical energy being inflicted. A unique type of wound results when heat or chemical energy rather than mechanical injury occurs as there is no mechanical disruption and there is no introduction of foreign material, yet there is a disruption in the chemical energy storage mechanism used for normal metabolism of cells and tissue health.

The human body responds to any wounding event with a predictable sequence of events called the inflammatory process. The inflammatory process is completely controlled and mediated by the immune system and its programmed, cascading, sequence of multiple intra and extra cellular factors. These factors are released following an injury to signal next appropriate steps to stabilize any wound, to isolate and remove foreign proteins if they have been introduced, and to prepare the area for stages of healing to follow.

The processes occur creating a condition known as inflammation. Acute inflammation, as seen with injuries from heat causing first or second degree burns, begins within seconds to minutes following the injury of tissues and progresses over 48 to 72 hours. The classical four symptoms of inflammation after an injury were described in writings by the Roman encyclopedist, Celsus, in the year 0004 A.D. over two thousand years ago. Those cardinal symptoms include: dolor (pain), tumor (swelling), rubor (redness), and calor (increased heat) at the site of the inflammation.

Pain occurs by direct signaling of mediators that incite pain neuroreceptors and from swelling in the ensuing hours creating pressure on those same receptors. Blood flow increases to the wound area to cause the redness from expanded vessels carrying oxygenated (red) blood. Capillaries dilate and allow leakage of blood serum out into the tissue spaces creating swelling. The increased permeability of the capillaries occurs because the endothelial cells separate from one another at their edges. With burns, the swelling increases over a period of 12-24 hours resulting in separation of the epidermis from the dermis to create a blister if inflammation is not controlled and the burn is severe enough.

Burn injuries differ somewhat from all other types/causes of injury in that an additional quantity of energy in the form of heat is the cause of the injury. Science reports effectively explain the “why and how” for each of the symptoms from burns EXCEPT for the observed elevated skin surface temperature. The ‘normal’ benchmark for body temperature was established by a 19th century German physician called Dr Carl Wunderlich. He is credited with taking temperature readings from thousands of patients, which led him to propose that 37 C was normal body temperature. It is generally accepted that normal body temperature ranges between 36.1 C (97 F) to 37.2 C (99 F).

Acute sunburned skin exhibits an increased surface temperature of above the normal body core temperature of 98.6 F degrees for the first 36 hours. All literature found simply mentions that the increase in skin surface heat is from an increased blood flow from core temperature blood to the skin after injury. This diversion or redirection of flow certainly occurs, but the increased blood flow could not increase skin heat to higher than core body temperature that averages 98.6 degrees.

All burns are a type of wound. They occur as a result of energy, often but not always energy in the form of heat, applied in an amount and over a period of time sufficient to cause injury to the skin and/or deeper tissues. Thermal burns occur from the skin touching or being in contact with a significantly hot item or surface such as a hot stove top or pan. Radiation burns occur as a result of penetration of energy into the skin from a source of radiation energy.

The wavelength of the radiation energy and the quantity of energy in the radiation beam are defined by the source of radiation energy. The wave type (wavelength) and the quantity of energy delivered are significant into the depth of penetration and where the energy is ultimately deposited. Beneficial radiation sources include X-rays, gamma rays, etc and are designed to penetrate deeper than the skin for cancer treatments, etc. As therapeutic radiation rays are not always “pure wavelength” some scattering of energy may be absorbed in the skin during treatments. Thus we see skin injury changes often in association with radiation therapy directed to deeper tissues.

By far, the most common radiation energy source causing burn injury world-wide is the sun, specifically the ultraviolet ray (UV) spectrum.

Standard medical classification of burn severity includes designations of first, second, or third degree based on the depth of the burn in the skin. 1st degree burns involve only the epidermal layer of skin. This is the layer of skin that separates to form the external envelope, or capsule, that is filled with blister fluid seen after some burns. The epidermis contains living cells in the basal layer that migrate over time and die a natural death before eventually being shed naturally due to wear and exposure to external rubbing forces on the skin.

The epidermis contains no blood vessels or nerve fibers, but is intimately connected to certain sensory receptor mechanisms for nerve signals. Supply of oxygen and nutrients to the epidermal living cells and removal of waste products of metabolism occurs by transfer via the extracellular fluid to and from the dermis where capillaries exist. The transfers are sometimes passive due to osmotic gradients, but include active transport requiring biological pumping mechanisms dependent on chemical energy supplied by well known mechanisms

Second degree burns involve both epidermis and the next deeper layer of skin, the dermis. This is the burn type that can heal without skin grafting and includes the majority of burns experienced in human life and the type of burns to be successfully treated using this method. Full thickness burns sustained at the time of/during the injury in a short period of time would be full thickness due to the severe amount of energy and the exposure time of the skin. These burns cause injury that will require surgical repair to heal are often caused by only seconds, or at the most minutes, of exposure time.

Sunburn can occur in less than 15 minutes, and in seconds when exposed to non-shielded welding arcs or other sources of intense ultraviolet light. Nevertheless, the inflicted harm is often not immediately obvious.

After the UV exposure, skin may turn red in as little as 30 minutes but most often takes 2 to 6 hours. In untreated skin with sunburns, the red color normally lasts for 5-days. Pain normally begins 2-4 hours after the UV exposure period and is usually most extreme 6 to 48 hours after exposure. The burn injury continues to develop for 24 to 72 hours, and blisters are noted by 24-36 hours. Blistered and non-blistered skin is usually followed by peeling skin in 3 to 8 days. Some peeling and itching may continue for several weeks.

b. Energy Transfers

Living organisms exist in a dynamic steady state, never at equilibrium with their surroundings with an ongoing flow of electrons that provides energy for metabolism of the organism. Photosynthetic cells in plants absorb light energy and use it to drive electrons from water to carbon dioxide forming energy of rich products, while releasing O2 into the atmosphere. Six carbon dioxide molecules plus six water molecules and visible light yield one molecule of glucose. Thus, plant photosynthesis is a light-driven reduction of carbon dioxide.

In animal systems, glucose is the energy supply from foods that can then yield energy for metabolism, breathing, muscle contraction, and all cellular chemical reactions requiring energy input.

Virtually all energy transductions in cells can be traced to this flow of electrons from one molecule to another in a “downhill” flow from higher to lower electrochemical potential. As such, this is formally analogous to the flow of electrons in a battery-driven electric circuit. All these reactions involving electron flow are oxidation-reduction, Redox, reactions. One reactant is oxidized, loses electrons, as another one is reduced, gains, electrons.

c. Lactate in the Body

Normal plasma lactate, in the blood, concentration in humans is 0.3-1.3 mmol/liter. Skin lactate concentration is normally nearly twice plasma lactate concentration at 2.48+/−0.17 mmol/liter. Redox mechanisms involving the enzyme Lactic Dehydrogenase, ATP/ADP conversion, NAD/NADH conversions, and Lactic acid conversion reaction into pyruvate, are all involved as major players in the bioenergetics and transfer of energy to and from cellular components and enzyme systems.

Thus, there are significant inflammatory responses occurring with any wound injury. Our findings relate to cellular activity within damaged cells and tissue in a less than full thickness skin energy injury that paradoxically affects cellular reaction to the energy injury.

Patents disclosing information relevant to lactic acid application on the skin include: U.S. Pat. No. 5,716,625, issued to Hahn, et al. on Feb. 10, 1998 entitled Formulations and methods for reducing skin irritation, which describes lactic acid as an exfoliant and skin irritant; U.S. Pat. No. 6,630,163, issued to Murad on Oct. 7, 2003 entitled Method of treating dermatological disorders with fruit extracts, which teaches lactic acid as a moisturizer; U.S. Pat. No. 6,890,566, issued to Arquette on May 10, 2005 entitled Composition and method to whiten and exfoliate skin, discusses lactic acid as part of a skin whitener; U.S. Pat. No. 4,021,572, issued to Van Scott, et al. on May 3, 1977 entitled Prophylactic and therapeutic treatment of acne vulgaris utilizing lactamides and quaternary ammonium lactates, discusses using lactic acid as a reactant in order to form an acne treatment; and U.S. Pat. No. 5,877,212, issued to Yu, et al. on Mar. 2, 1999 entitled Molecular complex and control-release of alpha hydroxyacids, also discusses lactic acid being a skin irritant. Each of these patents is hereby expressly incorporated by reference in their entirety.

From this prior art knowledge, it may be seen that the prior art is very limited in its teaching and utilization, and an improved method for skin injury treatment is needed to overcome these limitations.

### SUMMARY OF THE INVENTION

The present invention is directed to an improved method for treating burn injuries having an associated inflammatory response of the skin using a low pH l-lactic acid solution. The method for impeding skin's reaction to the tissue energy injury works on the injury area on the skin, and is performed after the injury has occurred. The method includes providing and applying an energy injury treatment product to the injury area. The energy injury treatment product includes the essential ingredient of a l-lactic acid in a concentration sufficient to give the energy injury treatment product a pH less than four pH and preferably around 2.34 pH with a concentration preferably between 5 and 15% but allowable to the limits of effectiveness and irritation of the particular patient. The application is performed during the inflammatory response time and is preferably applied between the initiation of the inflammatory response up to around six hours after the response time. The method includes reapplying the product during any inflammatory response at appropriate intervals.

### BRIEF DESCRIPTION OF THE SEVERAL VIEWS OF THE DRAWINGS

In the following drawings, which form a part of the specification and which are to be construed in conjunction therewith, and in which like reference numerals have been employed throughout wherever possible to indicate like parts in the various views:

FIG. 1 is a chart view of a normal inflammatory response compared against a method using a single application of the l-lactic acid product.

FIG. 2 is a chart view of a normal inflammatory response compared against a method using multiple applications of the l-lactic acid product showing the increased effectiveness over the single application.

FIGS. 3a and 3b provide a comparison of a sunburned back before and after the treatment of the present invention.

FIGS. 4a and 4b provide a comparison of an oven rack burned thumb before and after the treatment of the present invention.

FIGS. 5a, 5b, and 5c provide a comparison of a scalding liquid burned face before, during, and after the treatment of the present invention.

FIG. 6 is a schematic representation of the citric acid cycle in the cell.

FIG. 7 is a schematic representation of ion movement across a membrane from high concentration to low concentration.

### DETAILED DESCRIPTION OF THE INVENTION

FIG. 1 shows the interruption and reduction of the inflammatory response 60 to an energy injury 50 by the application 100 of concentrated low ph L-lactic acid. An energy injury is a sunburn, heat burn, or other radiation energy burn including thermal heat energy burns. The term energy injury does not include mechanical trauma such as being hit with a stick. As noted by the prior art discussed above, L-lactic acid was originally intended to facilitate reversing the signs/appearance of aging in cosmetic surgery patients by loosening and subsequent exfoliation of outer layer dead skin cells using L-Lactic Acid as the active ingredient. L-Lactic Acid is the only stereoisomer configuration of lactic acid that is active in cellular metabolism. In its original application, the L-Lactic Acid formulation very effectively results in increased epidermal cell turnover and stimulation of new collagen deposition in the deeper dermal skin layer of uninjured skin. However, the prior art teaches that acid irritates and burns the skin and teaches that concentrated acid should never be applied to an injury or burn area. In contrast to the prior art teachings, FIG. 1 shows that a paradoxical result is seen when this formulation is applied topically to any heat-caused burn such as a deep second degree burn. As noted by the timeline for the impeded inflammatory response of FIG. 1, the product was applied within four hours of the initial burn and this resulted in an immediate and significant impediment to the skin inflammatory response when compared against the untreated norm. In this example, there was no apparent exfoliation or blister occurrence. In fact, no blister formation has occurred after this treatment in any burn cases. In this manner, the action of 15% or less L-Lactic Acid with pH of less than 3.0 applied topically to an acute severe second degree burn appears to safely halt and then reverse the dermal inflammatory response cascade of events. Thus, capillary swelling decreases, leaked vascular fluid returns into the capillaries and/or lymph channels, and redness disappears as a result of fewer red blood cells in the area.

The formula for this product used in the present invention is, by weight percentage:

| | |
|---|---|
| WATER | 40.000 |
| SD ALCOHOL 39 | 40.000 |
| PROPYLENE GLYCOL | 10.000 |
| L-LACTIC ACID | 10.000 |

A topical anesthetic can also be added to this formulation, with the preferred anesthetic being lidocaine with a concentration of 0.5 to 20%. In this formulation, SD Alcohol is simple ethanol that has been rendered unsuitable for drinking and is therefore not taxed as drinking alcohol, the chemical formula is CH₃CH₂OH with a molecular weight of 46.06844 g/mol. Water is just distilled water, the chemical formula is H₂O with a molecular weight of 18.01528 g/mol. Propylene glycol is simply to allow thorough mixing of all ingredients and is present in probably a majority of cosmetic/topical products, the chemical formula is C₃H₈O₂ with a molecular weight of 76.09442 g/mol. Thus, the difference in this formulation is the high concentration of L-lactic acid.

L-lactic acid, chemical formula is C₃H₆O₃ with a molecular weight of 90.07794 g/mol, is known to exist in small quantities and concentrations in every cell in the human body and has a multitude of roles. Lactic Acid is a unique molecule with a number of carbon molecules that allow it to be a mediator that can interact with energy at the cellular level. L-lactic acid is known to be one of the primary mediators of the cellular immune system. It stimulates an increase in tumor necrosis factor. It is normally present in highest concentrations in the skin itself as compared to the rest of the body. In contrast to the normally low concentrations found in skin, the preferred product formulation is based on 10% weight/weight concentration of L-Lactic Acid, with pH of 2.34 that is compared to normal skin lactate levels that are around <3 mmol/L and skin pH between 6.8-7.0 pH. For the lactic acid of the present invention, we have found that above a 4.0 ph the lactic acid appears to be ineffective for the present treatment. A 4.0 pH concentration is slightly effective, with an increased effectiveness as the L-lactic acid concentration is increased and the pH is lowered with a 3.5 pH concentration increasing effectiveness over the 4.0 pH, a 3.0 pH increasing effectiveness compared to the 3.5 pH, a 2.8 pH further increasing effectiveness, with increased effectiveness found at 2.5 pH, 2.4 pH, with an ideal occurring around 2.35 pH to 2.34 pH. The decreased pH of the formula is associated with an increased weight percentage of the L-lactic acid with the above formula having an ideal weight percentage concentration between five and fifteen percent, and the preferred embodiment using the ten percent noted above. Concentrations as high as 20 to 30 percent may be used, but the high ranges over 30% incite exfoliation and encounter patient safety and patient skin sensitivity that become limiting factors on the upper end of the concentrations.

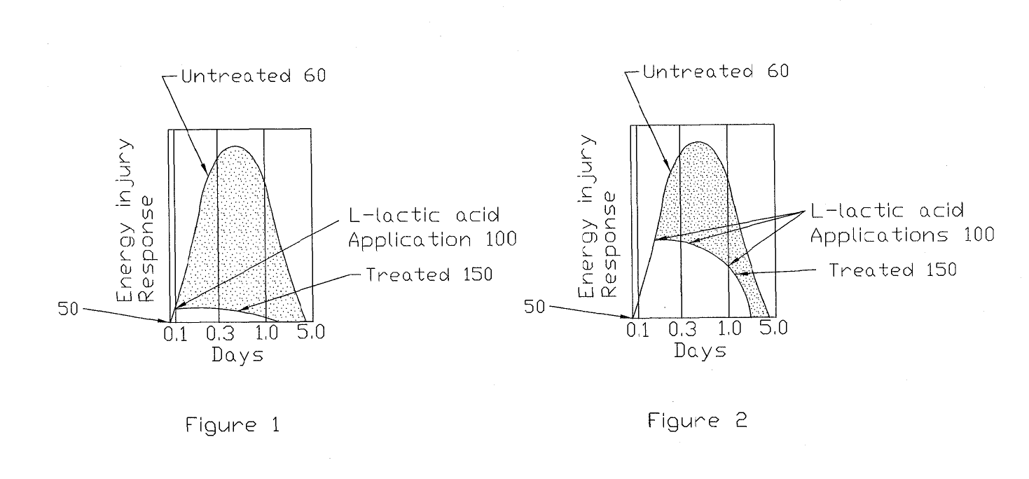

The preferred and proven application of the product should occur as soon as possible after the energy injury during the inflammatory response. However, the application is effective at any time during the skin inflammatory response. This initial application, if possible, should be within one hour for ANY first or second degree burn including a deep second degree burn such as a sunburn, touching a 400 degree oven pan. radiation therapy, or laser resurfacing treatment. This topical application has a phenomenal effect on both the visible appearance and experienced physical symptoms of the burn. We have successfully tested applications at 1 hour, 2 hours, 4 hours, and 6 hours after the energy injury. When looking at FIGS. 1 and 2 one may see that this initial application 100 time affects how much of the untreated skin inflammatory response 60 and damage that must be overcome with the faster the application 100, the better the treated effect 150. As may also be understood from FIG. 1, if applied 100 early after the burn injury 50, the expected progression of the inflammatory process 60 after an injury is impeded in the treated response 150 and the symptoms, including visual and physical symptoms already present at the time of L-Lactic Acid application are significantly reversed. FIG. 2 shows how the continued applications 100 are preferably done on a multiple-time-per-day schedule, with the preferred embodiment using twelve hour intervals, although 6 hour intervals can also be effective. The patient schedule, effectiveness of the application 100, and the increased concentration resulting in an increased patient's skin irritation become an issue with shortening the intervals for reapplication. FIG. 2 shows how the continued applications 100 continue to offset any additional reaction 60 and the treated response 150 appears to further decrease the normal time associated with the healing process.

The present method is effective on dealing with energy exposure injuries including both increased energy exposure and decreased energy exposure. These energy exposure injuries are injuries to skin that are not full thickness, and are caused by increased energy such as ultra violet light, radiation, or thermal heat coming into the skin and decreased energy exposure including frostbite and may work on other injuries such as trench foot, chilblains, etc. In addition to sunburn, ultraviolet radiation burns, thermal burns including hot iron, hot oven rack, grease burns, and hot liquid burns, the present invention is also believed to work on steam burns, flash burns, other radiation burns, chemical burns including chemical peels, and laser resurfacing injuries including those from CO2 or Fraxel.

The present invention teaches that with proper concentration and pH, the lactic acid conversion reaction from energy exposure is reversible. The present method reverses the normal skin injury reaction using L-Lactic Acid as a principal mediator of cellular metabolism and bioenergetics. It is believed that the L-lactic acid drives a reversal of the metabolically released heat energy back into a safely stored chemical energy form. This avoids the creation of free radicals or damage to proteins and/or DNA that lead to skin cancers and other problems. To understand the reversal of the heat energy damage cycle we begin with the first law of thermodynamics that states that energy cannot be created or destroyed, it can only be transformed into another kind of energy. The energy injury damages and can cook and/or break down the skin causing the initiation of the inflammatory responses. For these injuries, reversal of this can be done by transferring initial energy or inflammation energy back into chemical storage to prevent associated cellular damage compared to untreated sunburns. For this explanation, this is done through the topical application of the L-lactic acid in a concentration of 10% by weight having a ph of around 2.34 pH. Several patient examples are provided to illustrate the effectiveness of the present treatment.

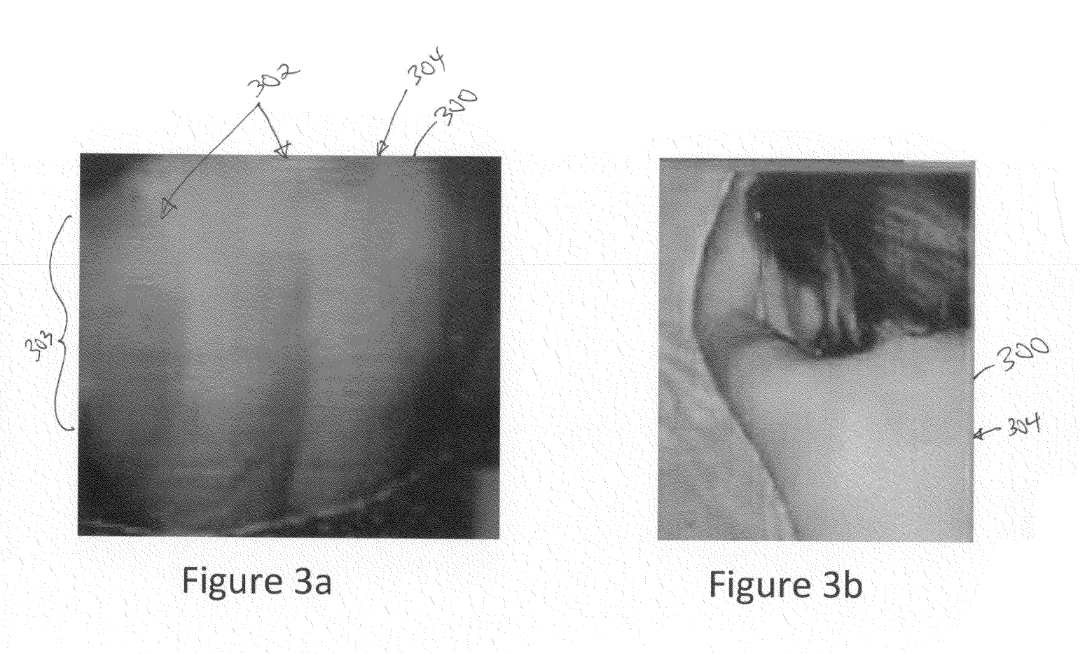

FIGS. 3a and 3b provide a comparison of a sunburn patient 300 showing a sunburn 302 on skin 304 causing a sunburn energy injury area 303. FIG. 3a shows the injury area 303 six hours after a severe sunburn 303 when the preferred formula concentration of 10% by weight, 2.34 pH L-lactic acid solution was initially applied. FIG. 3b shows the same patient 24 hours after the burn 303 with no visible redness or injury remaining on the skin 304.

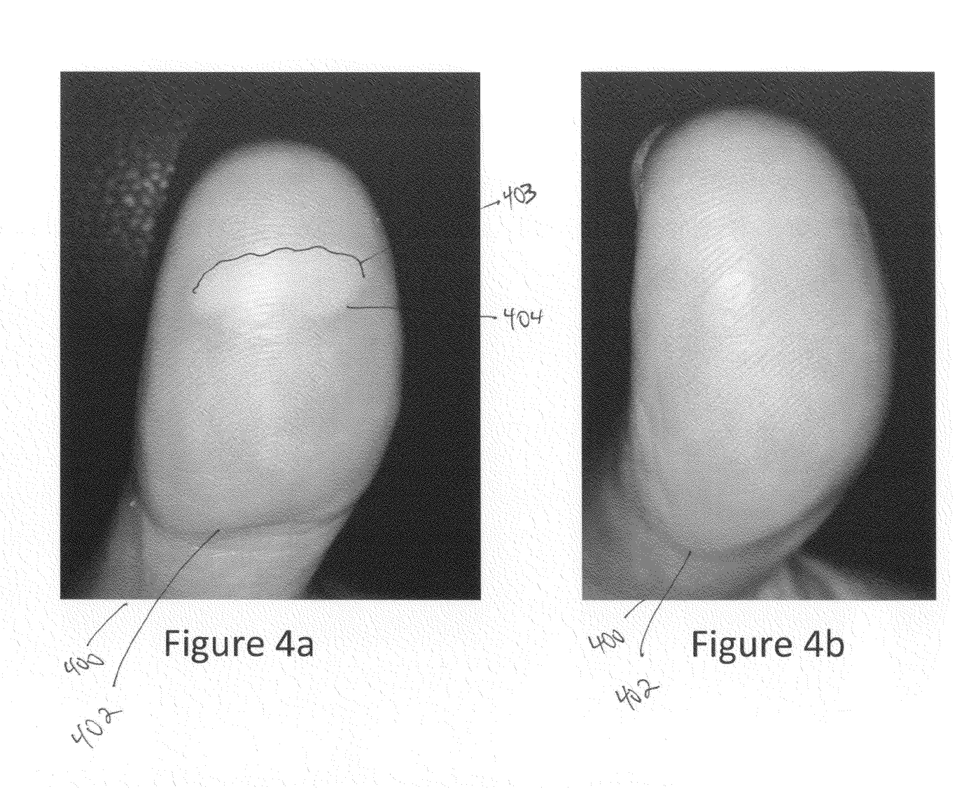

FIGS. 4a and 4b provide a comparison of an oven rack burn patient 400 showing a burn 403 from a four hundred degree oven rack touched with the skin 404 on a thumb 402. FIG. 4a shows the oven rack burn energy injury area 403 five minutes after the burn 403 occurred when the preferred formula concentration of 10% by weight, 2.34 pH L-lactic acid solution was initially applied. Pain stopped within 5 minutes of the application of the solution. The solution was applied twice daily for five days and FIG. 4b shows the same patient 400 on day five showing that no blister ever formed and the thumb 402 returning to normal skin 404 color within those five days.

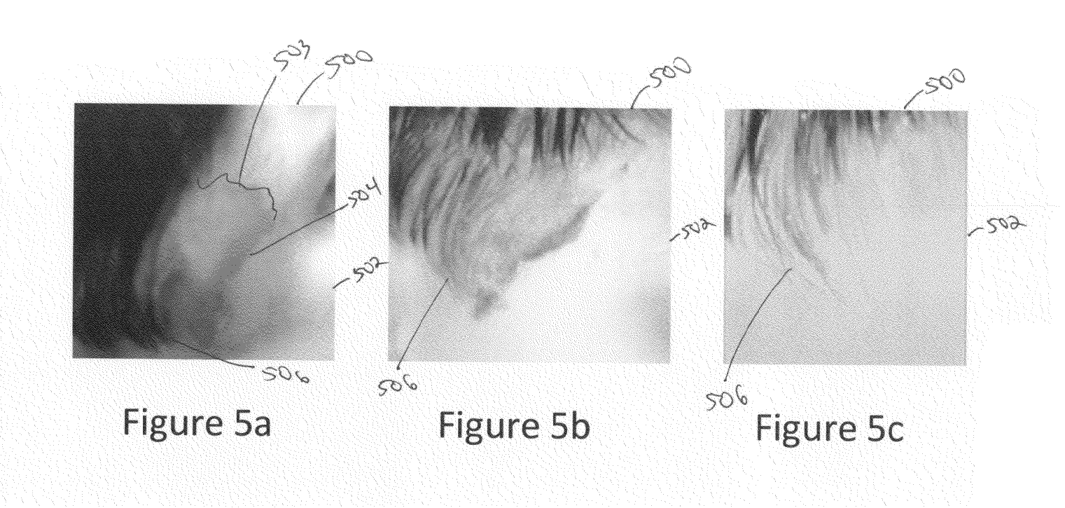

FIGS. 5a, 5b, and 5c provide a comparison of a scalding water patient 500 showing a boiling water burn 503 on the skin 504 of the facial tissue 502 at the hairline 506. FIG. 5a shows the initial boiling water energy injury 503 when the preferred formula concentration of 10% by weight, 2.34 pH L-lactic acid solution was initially applied. The solution was applied twice daily and FIG. 5b shows the same patient 500 on day six again showing that no blister ever formed and FIG. 5c shows the lack of any scarring on the patient 500 at day 14.

Clinical notes regarding burn injuries including the following comments:

“completely neutralized all symptoms of a sunburn on all areas applied including a return to normal skin color, no redness, by 24 hours. Pain was absent within 1 hour after first application . . . comparison [area was still] very red, sore.”

“4 hours after . . . sunburn . . . drew the sting out immediately . . . no more discomfort . . . burn was completely gone with no sign of a burn by 72 hours.”

We know that the product and method work for this purpose, and have an operating theory on how this works. We believe that the energy harm caused by the injury and the inflammatory process is reversed by the low pH and high concentration of the l-lactic acid. The following information is provided for consideration in relation to this reversed energy theory.

Energy Transfer:

Based on the available evidence, it is extremely plausible, if not probable, that the enzyme, ATP synthase, and oxidative phosphorylation along with Lactic Dehydrogenase are also likely to be a major components in the redistribution of original heat energy driven into the skin during the injury being converted into “stored” chemical energy. ATP is the most commonly used “energy currency” of cells. ATP, adenosine triphosphate, is formed from ADP, adenosine diphosphate, plus Pi, inorganic phosphate. An input of energy is required during the time ADP and Pi are joined together by the ATP synthase enzyme.

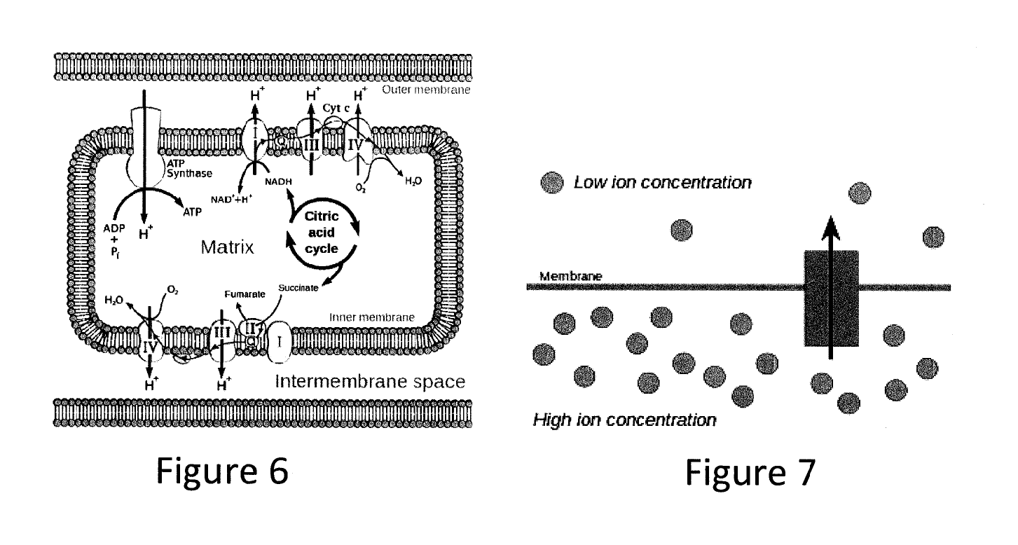

This energy transfer most likely comes from hydrogen ions, H+, in the form of a proton readily supplied by the applied lactic acid in the treatment. It is known that Lactic Acid easily passes into cells reversibly and is highly dissociated into H+ and lactate−. We know that in the electron flow of energy in oxidative phosphorylation we see the overview of the energy transfer in FIG. 6 showing the intracellular mitochondria.

The energy flow works by using energy-releasing chemical reactions to drive energy-requiring reactions: The two sets of reactions are said to be coupled. This means one cannot occur without the other. The flow of electrons through the electron transport chain, from electron donors such as NADH to electron acceptors such as oxygen, is an exergonic process—it releases energy, whereas the synthesis of ATP is an endergonic process, which requires an input of energy.

Chemiosmosis is the movement of ions across a selectively permeable membrane, such as exist in mitochondria within human cells, shown in FIG. 7, down their electrochemical gradient. More specifically, it relates to the generation of ATP by the movement of hydrogen ions across a membrane during cellular respiration. Thus, an Ion gradient has potential energy and can be used to power chemical reactions when the ions pass through a channel as shown in FIG. 7.

Hydrogen ions, protons, will diffuse from an area of high proton concentration to an area of lower proton concentration. Peter Mitchell proposed that an electrochemical concentration gradient of protons across a membrane could be harnessed to make ATP. He linked this process to osmosis, the diffusion of water across a membrane, which is why it is called chemiosmosis.

ATP synthase is the enzyme that makes ATP enabled by chemiosmosis. It allows protons to pass through the membrane and uses the kinetic energy thus available to phosphorylate ADP, making ATP. The generation of ATP by chemiosmosis occurs in both chloroplasts and mitochondria as well as in most bacteria and archaea.

Both the electron transport chain and the ATP synthase are embedded in an intracellular mitchondrial membrane. Energy is transferred from the electron transport chain to the ATP synthase by movements of protons across this membrane, in a process called chemiosmosis. In practice, this is like a simple electric circuit, with a current of protons being driven from the negative N-side of the membrane to the positive P-side by the proton-pumping enzymes of the electron transport chain. These enzymes are like a battery, as they perform work to drive current through the circuit. The movement of protons creates an electrochemical gradient across the membrane, which is often called the proton-motive force. It has two components: a difference in proton concentration, H+ gradient or ΔpH, and a difference in electric potential, with the N-side having a negative charge.

This H+ gradient, ΔpH, is drastically changed by the topically applied lactic acid with pH of 2.34 that is drastically significant from the normal skin pH of 6.8-7.0. This apparently seems to drive the reactions backwards to reform ATP which requires energy, in the form of heat energy being given off from the burn, taken into the chemical action. The resultant reversal of the clinical signs of the inflammatory reaction in progress somehow is also reversed. Multiple events would have been set in motion from this sequence of energy transference. See FIG. 6 to understand the overview level of this process.

All of the biological chemical reactions involved are facilitated by specific key enzymes. One key factor affecting the rate of a reaction catalyzed by an enzyme is the concentration of the substrate. Two other critical factors for both the rate and the direction of a chemical reaction are temperature, and pH, determined by hydrogen ion concentration.

This may also be considered with the fact that the biological energy transformations obey the laws of thermodynamics. Many quantitative observations made by physicists and chemists on the inter-conversion of different forms of energy led, in the nineteenth century, to the formulation of two fundamental laws of thermodynamics. The first law is the principle of the conservation of energy: for any physical or chemical change, the total amount of energy in the universe remains constant; energy may change form or it may be transported from one region to another, but it cannot be created or destroyed. The second law of thermodynamics, which can be stated in several forms, says that the universe always tends toward increasing disorder: in all natural processes, the entropy of the universe increases.

Also to be considered is that the standard free-energy change is directly related to the equilibrium constant. The composition of a reacting system, a mixture of chemical reactants and products, tends to continue changing until equilibrium is reached. At the equilibrium concentration of reactants and products, the rates of the forward and reverse reactions are exactly equal and no further net change occurs in the system. The concentrations of reactants and products at equilibrium define the equilibrium constant, Keq. In the general reaction aA+bB cC+dD, where a, b, c, and d are the number of molecules of A, B, C, and D participating, the equilibrium constant is given by

Keq=[C]c[D]d/[A]a[B]b

where [A], [B], [C], and [D] are the molar concentrations of the reaction components at the point of equilibrium. When a reacting system is not at equilibrium, the tendency to move toward equilibrium represents a driving force, the magnitude of which can be expressed as the free-energy change for the reaction, AG.

Under standard conditions, 298 K=25° C., when reactants and products are initially present at 1 M Concentrations or, for gases, at partial pressures of 101.3 kilopascals, kPa, or 1 atm, the force driving the system toward equilibrium is defined as the standard free-energy change, AG°. By this definition, the standard state for reactions that involve hydrogen ions is [11+]=1M, or pH 0.

Most biochemical reactions in the body, in vivo, occur in well-buffered aqueous solutions near pH 7; both the pH and the concentration of water, 55.5 M, are essentially constant. For convenience of calculations, biochemists therefore define a different standard state, in which the concentration of H+ is 10-7 M, pH 7, and that of water is 55.5 m; for reactions that involve Mg2+, including most in which ATP is a reactant, its concentration in solution is commonly taken to be constant at 1 mm. Human biological reactions equations assume a body temperature of 37 degrees Celcius, 98.6 degrees Farenheight. The temperature above is due to excess heat energy and thus affects the equation equilibrium constants for a chemical formula reaction as well.

Physical constants based on this biochemical standard state are called standard transformed constants and are written with a prime, such as AG¹⁰ and K′_{eg} to distinguish them from the untransformed constants used by chemists and physicists. Notice that most other textbooks use the symbol AG⁰¹ rather than AG¹⁰. Our use of AG¹⁰, recommended by an international committee of chemists and biochemists, is intended to emphasize that the transformed free energy G′ is the criterion for equilibrium. By convention, when H2O, H+, and/or Me+ are reactants or products, their concentrations are not included in equations such as Equation 13-2 but are instead incorporated into the constants K′_{eg} and AG¹⁰.

Another item for consideration is catabolism that is the degradative phase of metabolism in which organic nutrient molecules, carbohydrates, fats, and proteins, are converted into smaller, simpler end products, such as lactic acid, CO2, NH3. Catabolic pathways release energy, some of which is conserved in the formation of ATP and reduced electron carriers: NADH, NADPH, and FADH2; the rest is lost as heat. In anabolism, also called biosynthesis, small, simple precursors are built up into larger and more complex molecules including lipids, polysaccharides, proteins, and nucleic acids. Anabolic reactions require an input of energy, generally in the form of the phosphoryl group transfer potential of ATP and the reducing power of NADH, NADPH, and FADH2.

Finally, we also note that ATP hydrolysis per se usually accomplishes nothing but the liberation of heat, which cannot drive a chemical process in an isothermal system. A single reaction arrow typically shown almost invariably represents a two-step process in which part of the ATP molecule, a phosphoryl or pyrophosphoryl group or the adenylate moiety, AMP, is first transferred to a substrate molecule or to an amino acid residue in an enzyme, becoming covalently attached to the substrate or the enzyme and raising its free-energy content. Then, in a second step, the phosphate-containing moiety transferred in the first step is displaced, generating Pi, PPi, or AMP. Thus ATP participates covalently in the enzyme-catalyzed reaction to which it contributes free energy.

Reference numerals used throughout the detailed description and the drawings correspond to the following elements:
burn injury 50
untreated skin inflammatory response 60
initial application 100
treated effect 150
sunburn patient 300
sunburn 302
sunburn injury area 303
skin 304
oven rack burn patient 400
thumb 402
oven rack burn injury area 403
hot metal burned skin 404
scalding water patient 500
facial tissue 502
boiling water burn 503
water burned skin 504
hairline 506

From the foregoing, it will be seen that this invention well adapted to obtain all the ends and objects herein set forth, together with other advantages which are inherent to the structure. It will also be understood that certain features and subcombinations are of utility and may be employed without reference to other features and subcombinations. This is contemplated by and is within the scope of the claims. Many possible embodiments may be made of the invention without departing from the scope thereof. Therefore, it is to be understood that all matter herein set forth or shown in the accompanying drawings is to be interpreted as illustrative and not in a limiting sense.

When interpreting the claims of this application, method claims may be recognized by the explicit use of the word ‘method’ in the preamble of the claims and the use of the ‘ing’ tense of the active word. Method claims should not be interpreted to have particular steps in a particular order unless the claim element specifically refers to a previous element, a previous action, or the result of a previous action. Apparatus claims may be recognized by the use of the word ‘apparatus’ in the preamble of the claim and should not be interpreted to have ‘means plus function language’ unless the word ‘means’ is specifically used in the claim element. The words ‘defining,’ ‘having,’ or ‘including’ should be interpreted as open ended claim language that allows additional elements or structures. Finally, where the claims recite “a” or “a first” element of the equivalent thereof, such claims should be understood to include incorporation of one or more such elements, neither requiring nor excluding two or more such elements.

1. A method for impeding skin's reaction to a tissue energy injury covering an injury area on skin, the tissue energy injury occurring at an injury time, the method comprising:
providing an energy Injury treatment product, the energy injury treatment product including l-lactic acid and a carrier selected from a carrier group Including water, alcohol and glycol, the energy injury treatment product having a pH of less than four pH; and
applying the energy injury treatment product to the injury area.

2. The method of claim 1, the energy injury treatment product having a ph of less than three point five pH.

3. The method of claim 1, the energy injury treatment product having a ph of less than three pH.

4. The method of claim 1, the energy injury treatment product having a ph of less than two point eight pH.

5. The method of claim 1, the energy injury treatment product having a ph of less than two point six pH.

6. The method of claim 1, the energy injury treatment product having a ph of less than two point five pH.

7. The method of claim 1, the energy injury treatment product having a ph of less than two point four pH.

8. The method of claim 1, the injury treatment product having a pH of two point three four pH.

9. The method of claim 1, the energy injury treatment product including a topical anesthetic.

10. The method of claim 1, applying further comprising: applying the injury treatment product within six hours of the injury time.

11. The method of claim 1, applying farther comprising: applying the injury treatment product within four hours of the injury time.

12. The method of claim 1, applying further comprising: applying the injury treatment product within two hours of the injury time.

13. The method of claim 1, applying further comprising: applying the injury treatment product within one hour of the injury time.

14. The method of claim 1, applying further comprising: reapplying the injury treatment product to the injury area.

15. The method of claim 1, applying further comprising: reapplying the injury treatment product to the injury area every twenty four hours until healed.

16. The method of claim 1, applying farther comprising: reapplying the injury treatment product to the injury area multiple times per day.

17. The method of claim 1, applying farther comprising: reapplying the injury treatment product to the injury area at less than twelve hour intervals.

18. A method for impeding skin's reaction to a tissue energy injury covering an injury area on skin, the tissue energy injury occurring at an injury time, the method comprising:
providing an energy injury treatment product, the energy injury treatment product including l-lactic acid and a carrier selected from a carrier group Including water, alcohol and glycol, the l-lactic acid in an amount greater than five percent by weight;
applying the injury treatment product to the skin.

19. The method of claim 18, the concentration being less than fifteen percent by weight.

20. The method of claim 18, the concentration being approximately ten percent by weight.
